(19) United States
(12) Patent Application Publication
(10) Pub. No.: US 2026/0274881 A1
(43) Pub. Date: Sep. 17, 2026

(54) LIQUID-PHASE PROCESS FOR PREPARING OLIGONUCLEOTIDES

(71) Applicant: EXACTMER LIMITED, Harrogate (GB)

(72) Inventors: Andrew Livingston, Harrogate (GB); Piers Robert James Gaffney, Harrogate (GB); Anna Livolsi, Harrogate (GB)

(21) Appl. No.: 19/473,675

(22) PCT Filed: Apr. 10, 2024
(86) PCT No.: PCT/GB2024/050937

§ 371 Date: Oct. 8, 2025

(30) Foreign Application Priority Data
Apr. 11, 2023 (GB) 2305283.0

(51) Int. Cl. C07H 1/06; C07H 1/02; C07H 21/00

(52) U.S. Cl. CPC C07H 1/06; C07H 1/02; C07H 21/00

(57) ABSTRACT

A membrane filtration-assisted, liquid-phase process for preparing oligonucleotides is described. The process addresses the disadvantages associated with the preparation, purification and storage of nucleoside phosphoramidites. The process of the invention allows for supported oligonucleotides of high purity to be grown from their constituent nucleosides in a stepwise manner, using simple nucleosides or short-chain oligonucleotides as the monomeric building blocks that are sequentially coupled to the supported growing oligonucleotide.

### INTRODUCTION

The present invention relates to a liquid-phase process for preparing oligonucleotides. More particularly, the present invention relates to a membrane filtration-assisted, liquid-phase process for preparing oligonucleotides.

### BACKGROUND OF THE INVENTION

Oligonucleotides (oligos) are short sequence-defined polymers of nucleotides (a.k.a. nucleoside phosphates). The structure of oligos typically consists of a ribose phosphate backbone, usually linking the 3′- and 5′-oxygen substituents of the ribose sugar via a phosphodiester unit, and each ribose ring has an aromatic nucleobase bound to the 1′-C of ribose. The combination of ribose sugar and nucleobase is called a nucleoside, and the nucleobase varies from one monomer unit to the next in natural and medicinal sequences. It is the nucleobase sequence that confers target specificity on oligos.

Oligonucleotide-based drugs are now being advanced as a new generation of therapeutics, functioning at the protein expression level, and have recently been validated as a new pharmaceutical modality for treating a wide range of serious or life-threatening indications. Oligo drugs differ chemically from natural oligos in that they bear chemical modifications at multiple sites, principally to increase stability in the body and to improve targeting. However, similarly to natural oligos, it is the precise sequence of nucleobase side-chains that defines the oligonucleotide's pharmaceutical function.

In pharmaceutical oligos the modifications commonly include replacing oxygen with sulfur on the phosphate backbone, placing substituents on the 2′-C of ribose (e.g. MeO, F, methoxyethyloxy (Moe)), constraining the configuration of the ribose sugar with extra rings of atoms, methylating or fluorinating nucleobases, and replacing ribose with another heterocycle, such as morpholine. Even so, drugs containing such modifications are still recognisable as oligonucleotides.

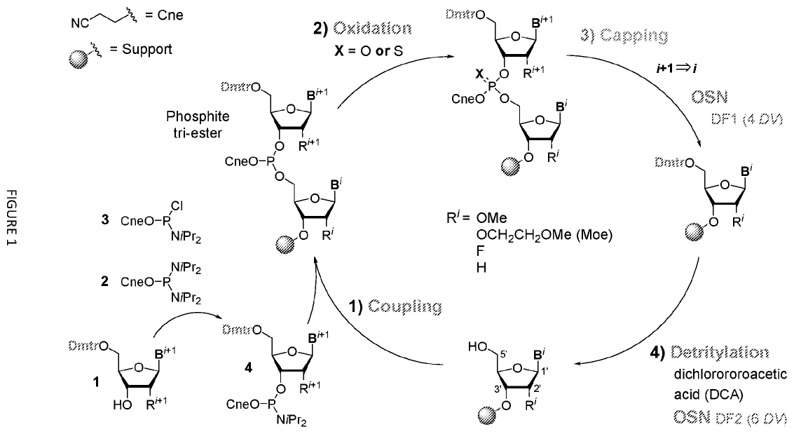

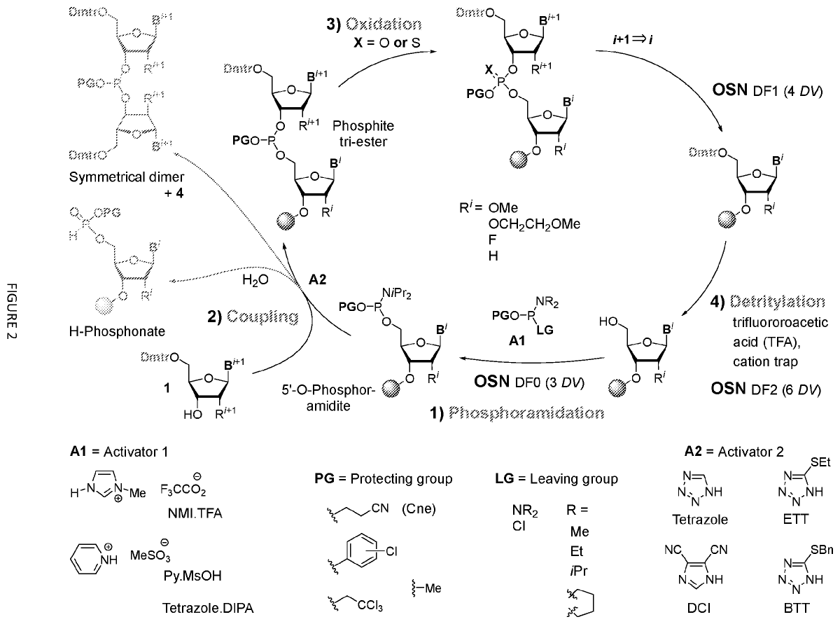

For many years oligonucleotides have been prepared using solid-phase oligonucleotide synthesis (SPOS) wherein a growing oligonucleotide is tethered to an insoluble solid support and grown by flowing reactive nucleoside phosphoramidite building blocks (4) over the insoluble solid support, as depicted in FIG. 1. Although this method has been the industry standard for decades, there are a number of drawbacks associated with SPOS. In particular, an excess of phosphoramidite (4) is commonly required in order to drive the reaction to completion. On a small scale an excess of many 10s of equivalents is common, thus adding to the significant cost of SPOS. Moreover, scaling up of SPOS reactions is limited to producing only ~15 kg of oligonucleotide per batch. This is particularly undesirable for their use as a pharmaceutical modality, wherein tonnes of oligonucleotide per annum would be required for a major medical indication (e.g. cardiovascular disease).

An alternative strategy to SPOS, which aims to address these scale-up and economic challenges, is liquid-phase oligonucleotide synthesis (LPOS). Indeed, liquid-phase reactions and liquid phase material handling are established technologies that can be performed at the multi-tonne scale, making LPOS a strong candidate for oligonucleotide preparation at scale. A typical approach to LPOS is to carry out sequential coupling reactions, adding monomers or multi-monomer oligomers (fragments) to a growing oligonucleotide in solution in a stepwise fashion, and then to use a suitable separation technology, such as membrane filtration, to separate unreacted monomers or fragments from the growing oligonucleotide (U.S. Pat. Nos. 8,664,357; 9,127,123; 10,239,996; EP3347402; Gaffney et al.; Kim et al.; So et al.; Yeo et al.; Dong et al.; WO 2016/188835 A1).

After the step of coupling a monomer or fragment onto a growing oligonucleotide, membrane filtration can be used to separate the unreacted monomers/fragments and any reaction debris from the growing oligonucleotide (Gaffney et al.; Kim et al.). The use of membrane filtration is therefore well-suited for LPOS, wherein a thorough purification step is essential after each coupling step.

However, when compared to typical small molecule drugs, oligos are very expensive to manufacture, both by classical SPOS and the developing field of LPOS. The largest fractions of the cost of oligo manufacturing by SPOS come from the nucleoside phosphoramidite (4), the amount of solvent required, the solid support, and the final purification of the crude oligo (Andrews et al.; Scozzari et al.). All other things being equal, reducing the cost significantly of any one of these factors would improve the economics of oligo production significantly.

Although membrane-assisted LPOS may be viewed as a more scalable alternative to SPOS, they are still hampered by the need for large quantities of expensive nucleoside phosphoramidite (4).

The Dmtr-nucleoside phosphoramidites (4) used in the SPOS manufacture of oligo pharmaceuticals are all prepared from the corresponding 5′-Dmtr-3′-hydroxy nucleosides (1), which have all the key features needed for synthesis already attached, except for the phosphorus moiety, including: (i) the critical temporary 5′-O-(4,4′-dimethoxytriphenylmethyl) (Dmtr) protecting group that is removed under mild acid conditions at the end of each chain extension cycle; (ii) permanent protecting groups that are only removed during the final global deprotection; (iii) ribose or other related sugar-like species; and (iv) a nucleobase.

There are a wide variety of routes to prepare 5′-Dmtr-nucleosides from a range of simpler precursors. Wherever possible, the isolation of intermediates along the synthetic pathway is performed by crystallisation or precipitation (assuming purification is required at all) to minimise costs and maximise scalability. Generally, the underlying nucleoside, including desired permanent modifications of both ribose and the nucleobase is constructed first, then the nucleobase is protected, and finally the Dmtr group is attached (EP0813539 B1; EP1612213 B1; Legorburu et al.; Liu et al.).

Still having regard to FIG. 1, a 5′-Dmtr-nucleoside may be converted to a 3′-phosphoramidite (Nielsen et al.; Pedersen et al.; Sanghvi et al.; Xie et al.), Nucleoside-OP(OPG)(NR₂), by reaction with either a chlorophosphine [(PGO)PCI(NR₂)] (3) or a bis-amidite [(PGO)P(NR₂)₂] (2), where PG (e.g. Cne in FIG. 1) is a permanent protecting group for the phosphotriester that will be carried through the entire oligo synthesis. The resultant phosphoramidite building block (4), with phosphorus in the P(III) oxidation state, is sensitive to deactivation by oxidation, including by oxygen in the air. If the phosphoramidite is exposed to any acid, it becomes extremely sensitive to hydrolysis, as well as loss of the 5′-O-Dmtr group. Due to these sensitivities commercial phosphoramidites are usually kept in refrigerated storage and transported by cold chain.

Before a phosphoramidite is committed to an oligo synthesis, it must first be purified. The main purpose is to remove any impurities that could participate in the chain extension cycle and lower the purity of the final oligo, known as a critical impurity. The most likely critical impurity from phosphoramidite synthesis is excess phosphoramidating reagent (e.g., (2) or (3)), which would permanently cap a growing oligo sequence if present in the phosphoramidite (4).

Unlike the Dmtr-nucleoside (1) and its precursors, the phosphoramidite building block (4) cannot usually be purified by crystallisation or precipitation. Although it will sometimes form a solid, instead of a gum, these processes tend to irreproducibly favour one of the two phosphoramidite diasteroisomers, and are not guaranteed to remove critical impurities. Then, when a phosphoramidite building block enriched in one diastereoisomer is subsequently employed in oligo chain extension, this can bias the diastereoisomeric ratio of the resultant linkage; this is particularly important if the phosphite tri-ester intermediate is then converted to a phosphorothioate analogue.

Consequently, Dmtr-nucleoside phosphoramidite building blocks (4) are almost always purified chromatographically through normal phase silica columns, usually using a gradient of anhydrous solvents such as ethyl acetate and heptane containing a small amount of organic base to ensure hydrolytic stability. Chromatography is not a favoured process-scale purification technique, since: (i) it is very difficult to scale up; (ii) it requires large amounts of flammable solvent; (iii) it is time consuming and requires careful monitoring; (iv) large fraction volumes must be screened and selected, combined and evaporated to dryness; and (v) it generates a lot of solid waste (the silica column). There have been attempts to simplify the purification of nucleoside phosphoramidites without chromatography (U.S. Pat. No. 7,960,542 B2), but they have not superseded this technique. Therefore, the complexity of the chromatographic purification process contributes significantly to the final cost of nucleoside phosphoramidites. Indeed, it is estimated to make the phosphoramidites (4) between 3- and 10-fold more expensive than their immediate nucleoside precursors (1).

The present invention was devised with the foregoing in mind.

### SUMMARY OF THE INVENTION

According to a first aspect of the present invention there is provided a liquid-phase process for forming an oligonucleotide, the process comprising the steps of:
a) providing a compound of formula I:

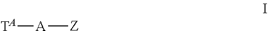

wherein A is a nucleoside or an oligonucleotide, T^{A} is a reactive terminal of the nucleoside or oligonucleotide, and Z is a soluble synthesis support to which the nucleoside or oligonucleotide is attached;
b) modifying the compound of formula I to form a compound of formula II:

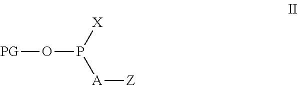

in which A and Z are as defined in respect of formula I, X is a tertiary amino group and PG is a protecting group;
c) isolating the compound of formula II by membrane filtration;
d) providing a compound of formula III:

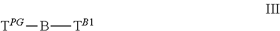

wherein B is a nucleoside or an oligonucleotide, T^{B1} is a reactive terminal of the nucleoside or oligonucleotide, and T^{PG} is a protected terminal of the nucleoside or oligonucleotide;
e) reacting the compound of formula II with the compound of formula III to form a compound of formula IV:

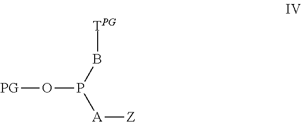

in which A and Z are as defined in respect of formula I, PG is as defined in respect of formula II, and B and T^{PG} are as defined in respect of formula III.

### DETAILED DESCRIPTION OF THE INVENTION

Throughout the entirety of the description and claims of this specification, where subject matter is described herein using the term “comprise” (or “comprises” or “comprising”), the same subject matter instead described using the term “consist of” (or “consists of” or “consisting of”) or “consist essentially of” (or “consists essentially of” or “consisting essentially of”) is also contemplated.

Throughout the description and claims of this specification, the singular encompasses the plural unless the context otherwise requires. In particular, where the indefinite article is used, the specification is to be understood as contemplating plurality as well as singularity, unless the context requires otherwise.

Features described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any of the specific embodiments recited herein. The invention extends to any novel one, or any novel combination, of the features disclosed in this specification (including any accompanying claims, abstract and drawings), or to any novel one, or any novel combination, of the steps of any method or process so disclosed.

Through rigorous investigations, the inventors have devised the liquid-phase process for forming an oligonucleotide according to the first aspect, which addresses the aforementioned disadvantages associated with the preparation, purification and storage of nucleoside phosphoramidites (4). The process of the invention allows for supported oligonucleotides of high purity to be grown from their constituent nucleosides in a stepwise manner, using simple nucleosides or oligonucleotides short-chain (and not nucleoside/oligonucleotide phosphoramidites, e.g., (4)) as the monomeric building blocks that are sequentially coupled to the supported growing oligonucleotide. Contrasting with the conventional, forward phosphoramidite coupling approach to LPOS, the process of the invention may be referred to herein as a reversed phosphoramidite coupling approach.

It will be understood that the term “nucleoside” used herein refers to a building block used in the formation an oligonucleotide, and typically comprises a nucleobase coupled to a ribose, backbone-forming heterocyclic moiety. In such backbone-forming heterocyclic moieties, oxygen atoms located at the 3′ and 5′ carbon atoms typically serve as the reactive terminals through which nucleosides can be coupled to one another. It is well known that ribose may be deoxygenated at the 2′ carbon. Additionally, as described hereinbefore, those of ordinary skill in the art will appreciate that synthetic nucleosides may contain various structural modifications, for example substitution at the 2′ carbon of ribose (e.g., with fluoro, methoxy or methoxyethyloxy), fusing one or more additional rings to ribose to constrain its configuration, bridging the ribose to constrain its configuration (e.g., as in Locked Nucleic Acid) and/or substitution on the nucleobase (e.g., with methyl or fluoro). Similarly, those of ordinary skill in the art will be familiar with variations of ribonucleosides comprising alternative backbone-forming heterocyclic moieties, such as in morpholine nucleosides, as well as the manner in which they are coupled to form oligos. It will be understood that such modifications and variations are embraced herein.

It will be understood that the term “oligonucleotide”, as used herein, refers to a chain of two or more nucleosides, wherein each pair of adjacent nucleosides in the chain is linked to one another, e.g., at the 3′ and 5′ positions of their ribose moieties, by a phosphorus-containing linker (e.g., a phosphodiester linkage, a methyl phosphonate linkage, a phosphorothioate linkage, a boranophosphate linkage, a phosphoramidate, or a mesyl phosphoramidate linkage). Typically, the term oligonucleotide used herein refers to at least 5 linked nucleosides, or at least 10 linked nucleosides.

The process may be performed in any suitable solvent. Suitably, the process is performed in acetonitrile, or in a solvent mixture comprising acetonitrile.

In compounds of formula I, A may be bound to Z at any chemically feasible position. In many instances, Z is attached to a backbone-forming heterocycle of A, such as at the 3′ or 5′ terminals of ribose. Suitably, the Z is attached to A at the 3′ terminal of ribose.

Typically, T^{A} and Z are disposed at opposing terminals of A. For example, when A is a nucleoside, Z may be attached at the 3′ terminal of ribose, with T^{A} disposed at the 5′ terminal of ribose, or vice versa. When A is an oligonucleotide, T^{A} and Z are disposed at opposing distal nucleosides. For example, Z may be attached at the 3′ terminal of ribose of one distal nucleoside, with T^{A} disposed at the 5′ terminal of ribose of the other distal nucleoside, or vice versa.

In many instances, T^{A} is disposed at the 5′ terminal of the nucleoside or oligonucleotide, A, and Z is disposed at the 3′ terminal of the nucleoside or oligonucleotide, A.

The compound of formula I may have a structure according to formula Ia:

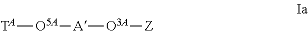

wherein
—O^{5A}-A′-O^{3A}— is a nucleoside or an oligonucleotide, in which O^{5A} is the oxygen located at the terminal 5′ carbon and O^{3A} is the oxygen located at the terminal 3′ carbon;
T^{A} is hydrogen; and
Z is a soluble synthesis support.

It will therefore be understood that the group —O^{5A}-A′-O^{3A}— in formula Ia may equate to the group A in formula I.

In step b), the compound of formula I is modified to yield a compound of formula II. The modification of the compound of formula I may be described as a phosphoramidation. Those of ordinary skill in the art will be familiar with the phosphoramidation of nucleosides.

X may be a group —NR₂, in which each R is independently selected from (1-6C)alkyl, or both R groups are linked such that when taken in combination with the nitrogen atom to which they are attached, they collectively form a 5- to 7-membered heterocycle. Suitably, each R is independently selected from (1-4C)alkyl, or both R groups are linked such that when taken in combination with the nitrogen atom to which they are attached, they collectively form a 5-membered heterocycle. More suitably, each R is independently selected from methyl, ethyl and isopropyl, or both R groups are linked such that when taken in combination with the nitrogen atom to which they are attached, they collectively form a pyrrolidinyl group. Most suitably, each R is isopropyl.

Those of ordinary skill in the art will be familiar with a variety of protecting groups suitable for use as PG. PG may be a base-labile protecting group. Examples of PG include methyl, isopropyl, tert-butyl, benzyl, allyl, phenyl, 2-chlorophenyl, 4-chlorophenyl, 3′5′-dimethoxybenzoin, p-hydroxyphenacyl, cyanoethyl, 9-fluorenylmethyl, 2-(trimethylsilyl)ethyl, 2-(methylsulfonyl)ethyl, 2-(benzenesulfonyl)ethyl, 4-nitrophenethyl, and 2,2,2-trichloroethyl. Suitably, PG is selected from the group consisting of cyanoethyl, 2-chlorophenyl, 4-chlorophenyl, 2,2,2-trichloroethyl and methyl. Most suitably, PG is cyanoethyl.

Step b) may comprise reacting the compound of formula I with a compound of formula A:

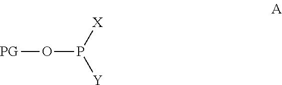

in which PG and X are as defined for the compound of formula II, and Y is halo or has any of those definitions outlined hereinbefore for X;
wherein when Y is not halo, step b) is conducted in the presence of a first phosphoramidite activator.

The compound of formula A may be described as a phosphoramidating agent, i.e., an agent used to convert a nucleoside into a nucleoside phosphoramidite. Those of ordinary skill in the art will be familiar with agents suitable for phosphoramidating nucleosides, and the manner in which they are used. For example, it will be understood by those of skill in the art that compounds of formula A in which Y is halo are typically used in the presence of a base, such as Et₃N or DIPEA.

Y may be Cl or any of those definitions outlined hereinbefore for X (e.g., —N(ⁱPr)₂).

Particularly suitable examples of the compound of formula A include:

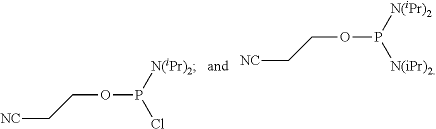

Phosphoramidite activators will be familiar to those of ordinary skill in the art. In particular, it will be understood that the first phosphoramidite activator is capable of reacting with the compound of formula A to form a compound of formula A′:

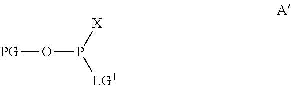

wherein X and PG are as defined for the compound of formula II, and
LG¹ is a leaving group;
and wherein the compound of formula A′ is capable of reacting with the compound of formula I to form the compound of formula II.

In many instances, LG¹ is an aza-heteroaryl. Suitably, LG¹ is imidazolyl, pyridyl or tetrazolyl. Particular, non-limiting examples of the first phosphoramidite activator are:

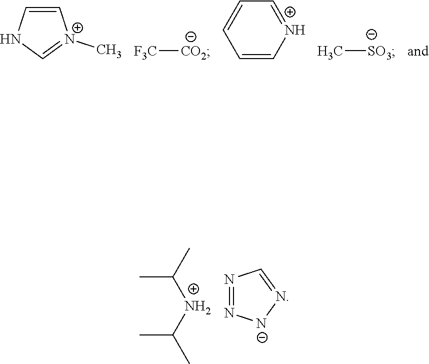

The first phosphoramidite activator may be denoted herein as A1.

Step b) is suitably conducted using an excess of the compound of formula A. This ensures the greatest possible conversion of the compound of formula I into the compound of formula II and avoids the occurrence of sequence deletion errors in the structure of the oligonucleotide being prepared.

When the compound of formula I is a compound of formula Ia, the compound of formula II may be a compound of formula IIa:

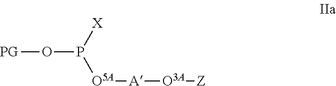

in which PG and X are as defined for formula II and —O^{5A}-A′-O^{3A}— and Z are as defined for formula Ia.

In step c), the compound of formula II is isolated by membrane filtration (e.g., membrane diafiltration). The compound of formula II may be isolated from one or more reactants used in step b). Such reactants may include the compound of formula I, the compound of formula A, the first phosphoramidite activator and/or the compound of formula A′. Removing or greatly reducing the quantity of residual compound of formula A before coupling step d) is important, since for every mole of the compound of formula A remaining, up to two moles of the compound of formula III may be undesirably consumed in the formation an unwanted symmetrical dimer.

The membrane used in step c) is suitably an organic solvent-resistant membrane. For example, the compound of formula II may be isolated by organic solvent nanofiltration. More suitably, the membrane is insoluble in the solvent(s) used in step b) (e.g., acetonitrile or a solvent mixture comprising acetonitrile). In such cases, the compound of formula II can be prepared and isolated in the same solvent(s).

Suitable membranes for use in isolating the compound of formula II (or any other compound described herein that is supported on Z) include polymeric membranes, ceramic membranes, and mixed polymeric/inorganic membranes. Membrane rejection Rᵢ is a common term known by those skilled in the art and is defined as:

$${R}_{i} = ( 1 - \frac{{C}_{Pi}}{{C}_{Ri}} ) \times 100 \% \qquad eq . ( 1 )$$

where C_{P,i}=concentration of species i in the permeate, permeate being the liquid which has passed through the membrane, and C_{R,i}=concentration of species i in the retentate, retentate being the liquid which has not passed through the membrane. It will be appreciated that a membrane is suitable for the invention if R_{(product)}>R_{(reactants)}, where product is the compound of formula II or any other compound described herein that is supported on Z).

The membrane may be formed from any polymeric or ceramic material which provides a separating layer capable of preferentially separating the compound of formula II from one or more reactants used in step b). In other words, the membrane will exhibit a rejection for the compound of formula II (or any other compound described herein that is supported on Z) that is greater than the rejection for the reactants used in step b. Suitably, the membrane is formed from or comprises a polymeric material suitable for fabricating microfiltration, ultrafiltration, nanofiltration or reverse osmosis membranes, including polyethylene, polypropylene, polytetrafluoroethylene (PTFE), polyvinylidene difluoride (PVDF), polysulfone, polyethersulfone, polyacrylonitrile, polyamide, polyester, polyimide, polyetherimide, cellulose acetate, polyaniline, polypyrrole, polybenzimidazole (PBI), polyetheretherketone (PEEK) and mixtures thereof. The membranes can be made by any technique known in the art, including sintering, stretching, track etching, template leaching, interfacial polymerisation or phase inversion. Membranes may be composite in nature (e.g., a thin film composite membrane) and/or be crosslinked or treated so as to improve their stability in certain solvents. PCT/GB2007/050218 and PCT/GB2015/050179 describe membranes which may be suitable for use as part of the invention. U.S. Pat. No. 10,913,033 describes a membrane that is particularly suitable for use as part of the invention.

Most suitably, the membrane is a crosslinked polybenzimidazole membrane (e.g., an integrally skinned, asymmetric, crosslinked polybenzimidazole membrane).

In compounds of formula III, T^{PG} and T^{B1} are disposed at opposing ends of B. For example, when B is a nucleoside, T^{B1} may be disposed at the 3′ terminal of ribose, with T^{PG} disposed at the 5′ terminal of ribose, or vice versa. When B is an oligonucleotide, T^{B1} and T^{PG} are disposed on opposing distal nucleosides. For example, T^{B1} may be attached at the 3′ terminal of ribose of one distal nucleoside, with T^{PG} disposed at the 5′ terminal of ribose of the other distal nucleoside, or vice versa.

In many instances, T^{B1} is disposed at the 3′ terminal of the nucleoside or oligonucleotide, B, and T^{PG} is disposed at the 5′ terminal of the nucleoside or oligonucleotide, B.

Having regard to T^{PG}, persons of ordinary skill in the art will be familiar with a variety of protecting groups used to prevent uncontrolled chain extension during the preparation of oligonucleotides. Typically, T^{PG} is a protecting group bound to the 5′ oxygen of B. In many instances, T^{PG} is an acid-labile protecting group, non-limiting examples of which include dimethoxytrityl (Dmtr/DMT), tert-butyl (tBu), tert-butyl oxycarbonyl (Boc), mono-methoxytriphenyl (Mmtr), triphenyl methyl (Tr), pentamethyl dihydrobenzofuran sufonyl (Pbf), Tetrahydropyranyl (Thp), tetrahydrofuranyl (Thf), para-methoxybenzyl (Pmb) and 2,4-dimethoxybenzyl. Most suitably, T^{PG} is dimethoxytrityl bound to the 5′ oxygen of B.

In many instances, the compound of formula III may have a structure according to formula Illa:

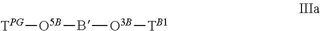

wherein
—O^{5B}—B′—O^{3B}— is a nucleoside or an oligonucleotide, in which O^{5B} is the oxygen located at the terminal 5′ carbon and O^{3B} is the oxygen located at the terminal 3′ carbon;
T^{B1} is hydrogen; and
T^{PG} is a protecting group (e.g., DMT).

It will therefore be understood that the group —O^{5B}—B′—O^{3B}— in formula Illa may equate to the group B in formula III.

In step e), the compound of formula II is reacted with the compound of formula III to afford a compound of formula IV. Persons of ordinary skill in the art will be able to select suitable conditions for performing the coupling reaction of step e).

Step e) is suitably conducted in the presence of a second phosphoramidite activator. Phosphoramidite activators will be familiar to those of ordinary skill in the art. In particular, it will be understood that the second phosphoramidite activator is capable of reacting with the compound of formula II to form a compound of formula II′:

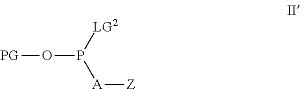

in which Z, A and PG are as defined for the compound of formula II;
LG² is a leaving group;
and wherein the compound of formula II′ is capable of reacting with the compound of formula III to form the compound of formula IV.

In many instances, LG² is aza-heteroaryl. Suitably, LG² is imidazolyl or tetrazolyl. Particular, non-limiting examples of the second phosphoramidite activator are tetrazole, 5-(ethylthio)-1H-tetrazole, 4,5-dicyanoimidazole and 5-(benzylthio)-1H-tetrazole. The second phosphoramidite activator may be denoted herein as A2.

When the compound of formula I is a compound of formula Ia, and the compound of formula III is a compound of formula Illa, the compound of formula IV may be a compound of formula IVa:

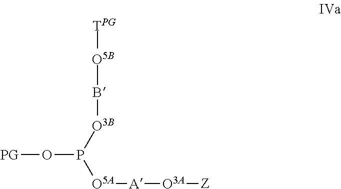

in which PG is as defined for formula II, —O^{5A}-A′-O^{3A}— and Z are as defined for formula Ia, and —O^{5B}—B′—O^{3B}— and T^{PG} are as defined in respect of formula Illa.

Step e) is suitably conducted under anhydrous conditions. It is at this point in the process for preparing an oligonucleotide that the presence of water may affect product purity and yield. Upon 5′-phosphoramidite activation, the presence of water may lead to formation of a 5′-H-phosphonate di-ester. The H-phosphonate can be converted to the corresponding phosphorothioate diester by modern sulfur transfer agents, such as xanthane hydride (XH) or PolyOrg Sulfa (POS), which can cap the oligo on which it occurs, thus leading to truncated impurities.

The process of the invention is notably less prone to unwanted side reactions than in conventional, forward phosphoramidite coupling approaches. As is well known in SPOS, if a high concentration of acidic phosphoramidite activator is present during the coupling reaction, this can lead to a low level of concomitant 5′-O-detritylation. The resultant newly exposed 5′-OH is then also extended leading to a n+1 defect, where two nucleoside building blocks add to one chain during the same coupling reaction. These are amongst the hardest impurities to separate from the final, full-length oligonucleotide. The process of the invention guards against such impurities. Isolating the compound of formula II (e.g., from residual compound of formula A) in step c) means that even if unwanted deprotection at T^{PG} (e.g., 5′-O-detritylation) occurs during step d), an n+1 defect cannot arise.

Z is a synthesis support that is soluble in the solvent(s) used for the process of the invention. Z may serve to retain the growing oligonucleotide chain in solution and/or confer molecular bulk to facilitate its isolation by membrane filtration. Z may, for example, be one or more of a branch point molecule, a polymer, a dendrimer, a dendron, a hyperbranched polymer, or an organic/inorganic material, including nanoparticles, fullerenes and 2-D materials such as graphene and boron nitride.

In many instances, Z is a polymeric soluble synthesis support (e.g., polyethylene glycol). Z may have a molecular weight (M_{w}) of 500-50,000 Da.

Z may be a compound of formula B:

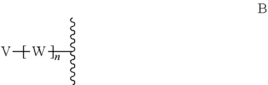

in which
V is an organic branch point,
W is a polymeric chain,
[symbol] represents a point of attachment to A, and
n is 1-8.

V will be understood to refer to a polyfunctional organic “hub” having a plurality of terminals to which polymeric chain(s), W, are attached. In many instances, V comprises fewer than 50 atoms in total, more suitably fewer than 20 atoms in total.

Each Wis a polymeric chain (e.g., polyethylene glycol) having a molecular weight (M_{w}) of 500-20,000 Da. More suitably, each W is a polymeric chain having a molecular weight (M_{w}) of 2000-15,000 Da. Most suitably, each Wis a polymeric chain having a molecular weight (M_{w}) of 8000-12,000 Da.

n may, for example, be 1-6. In many instances, n is 3-4.

Each W is bound to a nucleoside or oligonucleotide, A, as defined herein. Therefore, it will be understood that when Z is a compound of formula B, the process for forming an oligonucleotide yields n number of identical oligonucleotide chains.

It will be understood that each W may be attached directly to A, or attached indirectly to A via a linking moiety. A typical linking moiety will comprise fewer than 50 atoms in total, more suitably fewer than 30 atoms in total.

A particularly suitable example of a compound of formula B is:

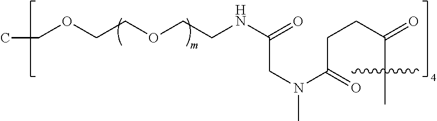

in which [symbol] represents the point of direct attachment to A (e.g., to the 3′ oxygen of A) and m is 100-500 (e.g., 200-300).

The process may further comprise a step of converting one of more P(III) phosphite triester linkages to a P(V) phosphotriester linkage, for example by oxidation or sulfur transfer. Such a step may be described generically herein as an oxidation step. Suitable reagents for performing this step will be familiar to those of ordinary skill in the art. As part of this step, the oxidation product (i.e., the compound comprising one or more P(V) phosphotriester linkages) may be isolated by membrane filtration. Membrane filtration may be conducted as described hereinbefore in relation to step c) (e.g., with the Z-tethered growing oligonucleotide being collected in the retentate and the oxidation step reactants and/or by-products collecting in the permeate).

The process may further comprise a step of contacting the oligonucleotide with a deprotecting agent to form a compound of formula V:

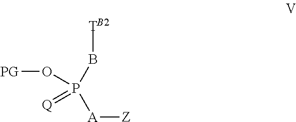

in which
Z and A are as defined in relation to formula I;
PG is as defined in relation to formula II;
Q is selected from absent, O or S; and
T^{B2} is a reactive terminal of B (e.g., a 5′ terminal).

The step of forming a compound of formula V may be described generically herein as the T^{PG} deprotection step. Suitable reagents for performing this step will be familiar to those of ordinary skill in the art. As part of this step, once formed, the compound of formula V may be isolated by membrane filtration. Membrane filtration may be conducted as described hereinbefore in relation to step c) (e.g., with the Z-tethered growing oligonucleotide being collected in the retentate and the T^{PG} deprotection step reactants and/or by-products collecting in the permeate).

It will be appreciated that where Q is absent, the P atom is in the P(III) state, whereas when Q is O or S, the P atom is in the P(V) state (i.e., following oxidation or sulfur transfer of the P(III) phosphite triester linkage).

It will be understood that the T^{PG} deprotection step (including any associated membrane filtration) occurs after step e), and before or after the oxidation step (including any associated membrane filtration). In many instances, the oxidation step (including any associated membrane filtration) occurs after step e) and before the T^{PG} deprotection step.

The sequence of steps a) to e), the oxidation step (including any associated membrane filtration) and the T^{PG} deprotection step (including any associated membrane filtration) may be repeated any number of times to grow, in a stepwise manner by incremental addition of nucleoside-containing building blocks, a supported oligonucleotide having a target length.

Thus, in many instances, the process may further comprise the steps of:
(i) providing a growing oligonucleotide, wherein the growing oligonucleotide is:
   A) the compound of formula V, or
   B) the compound of formula V, to which has been coupled one or more additional nucleosides, wherein the nucleoside most distant from Z has a reactive terminal;
(ii) modifying the growing oligonucleotide to form a compound of formula VI:

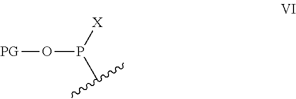

in which [symbol] represents a bond to the growing oligonucleotide (e.g., at the reactive terminal), and PG and X are as defined in relation to formula II;
(iii) isolating the compound of formula VI by membrane filtration;
(iv) providing a nucleoside or oligonucleotide building block, having a reactive terminal and a protected terminal;
(v) reacting the isolated compound of formula VI with the reactive terminal of the nucleoside or oligonucleotide building block to form a chain-extended oligonucleotide, the chain-extended oligonucleotide having a protected terminal;
(vi) isolating the chain-extended oligonucleotide by membrane filtration;
(vii) deprotecting the protected terminal of the isolated chain-extended oligonucleotide, and optionally repeating the sequence of steps (i) to (vii).

Where, for example, the reactive terminal of the growing oligonucleotide (i.e., the oligonucleotide provided in step (i) A) or B)) is the 5′ terminal, the reactive terminal of the nucleoside or oligonucleotide building block provided in step (iv) will be the 3′ terminal; in such cases, the protected terminal of the nucleoside or oligonucleotide building block provided in step (iv) will be the 5′ terminal. Where, for example, the reactive terminal of the growing oligonucleotide (i.e., the oligonucleotide provided in step (i) A) or B)) is the 3′ terminal, the reactive terminal of the nucleoside or oligonucleotide building block provided in step (iv) will be the 5′ terminal; in such cases, the protected terminal of the nucleoside or oligonucleotide building block provided in step (iv) will be the 3′ terminal. Persons of ordinary skill in the art will be familiar with the manner in which non-ribose-containing nucleosides (e.g., morpholino nucleosides) may be coupled.

The process comprising steps (i) to (vii) may further comprise a step of converting one of more P(III) phosphite triester linkages to a P(V) phosphotriester linkage (e.g., by oxidation or sulfur transfer). Most suitably, this is an intervening step performed between steps (v) and (vi). It will be understood that when step (vii) involves repeating the sequence of steps (i) to (vii), this includes any intervening steps.

The process may further comprise a step of removing the protecting group PG from one of more P(V) phosphotriester linkages. Such a step may be referred to herein as a global deprotection step. The global deprotection step may be performed once the desired length of oligonucleotide has been reached. As described hereinbefore, those of ordinary skill in the art will be familiar with those groups suitable for use as PG, as well as the techniques by which they can be removed.

The process may further comprise a step of cleaving the oligonucleotide from Z. Once the desired length of oligonucleotide has been reached, the soluble synthesis support may be removed. Those of ordinary skill in the art will be familiar with techniques by which this can be achieved.

The process of the invention is particularly well-suited to fragment-based oligo synthesis (e.g., where A and B are both oligonucleotides). The approach has advantages in that, being convergent, overall yields can be maximised and the purity of the full-length product is increased because each of the fragments is easier to prepare in high purity. Conventionally, before fragment coupling can be undertaken, two fragments must be separately prepared and isolated with all their protecting groups intact except for a chain terminal hydroxyl on each fragment to be coupled through a phosphate di-ester. This is almost always performed in solution phase, partly because fragments have slow diffusion constants for permeating into solid phase synthesis beads. One fragment must then be converted to a reactive phosphoramidite. This is a major challenge because the phosphoramidite fragment must be completely purified to remove any phosphoramidation reagent, which, if present during coupling to the other fragment, would successfully compete and cap the hydroxyl fragment. This latter issue can be readily avoided if the membrane assisted reversed phosphoramidite coupling approach of the invention is employed. The hydroxy terminal of a growing oligo is firstly phosphoramidated in solution and purified by membrane filtration. Purification can then be continued until the bifunctional P(III) reagent is reduced to a low concentration. Since the reactive oligo fragment has been prepared in solution where the rates of reaction of high molecular weight reactants are not reduced by phase transfer kinetics, there is no requirement to cleave the phosphoramidated fragment from the soluble support. The hydroxy fragment is then introduced into the reactor and the second phosphoramidite activator added to initiate coupling. A more open membrane may be selected for membrane filtration so that, after oxidation or sulfur transfer, the unsupported fragment may be separated from the ligated strands. This provides the opportunity to recover the valuable hydroxy fragment.

Another strength of the reversed phosphoramidite approach of the present invention is that it is entirely compatible with the established, or forward, phosphoramidite approach. Thus, if a given nucleoside is not available, but the corresponding nucleoside phosphoramidite is available, then the forward building block can be used to complete the chain extension. Forward and reversed coupling approaches can be swapped with each other as many times as is desired to build the desired sequence as the global deprotection step is unaffected by whether the forward or reversed approach is used to construct the fully protected full length product.

The process of the invention is suitably performed in a closed loop reactor. For example, the reactor may comprise a reaction vessel having an inlet and an outlet, and a membrane-containing purification vessel having an inlet, a retentate outlet and a permeate outlet, wherein the outlet of the reaction vessel is in fluid communication with the inlet of the purification vessel, and the retentate outlet of the purification vessel is in fluid communication with the inlet of the reaction vessel. Reaction steps (e.g., step b)) are conducted in the reaction vessel, after which the reaction medium is fed from the outlet of the reaction vessel to the inlet of the purification vessel for isolating (e.g., in step c)) the desired reaction product (e.g., the growing, supported oligonucleotide) in the retentate. The isolated reaction product then is fed from the retentate outlet to the reaction vessel inlet, whereby the isolated reaction product can partake in the next reaction step (e.g., step d)). The reactor may be described herein as a synthesiser.

The following numbered statements 1 to 57 are not claims, but instead describe particular aspects and embodiments of the invention:
1. A liquid-phase process for forming an oligonucleotide, the process comprising the steps of:
a) providing a compound of formula I:

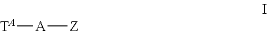

wherein A is a nucleoside or an oligonucleotide, T^{A} is a reactive terminal of the nucleoside or oligonucleotide, and Z is a soluble synthesis support to which the nucleoside or oligonucleotide is attached;
b) modifying the compound of formula I to form a compound of formula II:

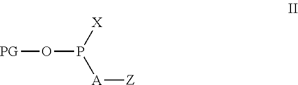

in which A and Z are as defined in respect of formula I, X is a tertiary amino group and PG is a protecting group;
c) isolating the compound of formula II by membrane filtration;
d) providing a compound of formula III:

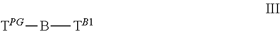

wherein B is a nucleoside or an oligonucleotide, T^{B1} is a reactive terminal of the nucleoside or oligonucleotide, and T^{PG} is a protected terminal of the nucleoside or oligonucleotide;
e) reacting the compound of formula II with the compound of formula III to form a compound of formula IV:

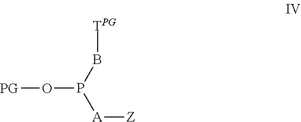

in which A and Z are as defined in respect of formula I, PG is as defined in respect of formula II, and B and T^{PG} are as defined in respect of formula III.
2. The liquid-phase process as defined in statement 1, wherein X is a group —NR₂, in which each R is independently selected from (1-6C)alkyl, or both R groups are linked such that when taken in combination with the nitrogen atom to which they are attached, they collectively form a 5- to 7-membered heterocycle.
3. The liquid-phase process as defined in statement 2, wherein each R is independently selected from (1-4C)alkyl, or both R groups are linked such that when taken in combination with the nitrogen atom to which they are attached, they collectively form a 5-membered heterocycle.
4. The liquid-phase process as defined in statement 2, wherein each R is independently selected from methyl, ethyl and isopropyl, or both R groups are linked such that when taken in combination with the nitrogen atom to which they are attached, they collectively form a pyrrolidinyl group.
5. The liquid-phase process as defined in statement 2, wherein each R is isopropyl.
6. The liquid-phase process as defined in any one of the preceding statements, wherein PG is a base-labile protecting group.
7. The liquid-phase process as defined in any one of the preceding statements, wherein PG is selected from the group consisting of methyl, isopropyl, tert-butyl, benzyl, allyl, phenyl, 2-chlorophenyl, 4-chlorophenyl, 3′5′-dimethoxybenzoin, p-hydroxyphenacyl, cyanoethyl, 9-fluorenylmethyl, 2-(trimethylsilyl)ethyl, 2-(methylsulfonyl)ethyl, 2-(benzenesulfonyl)ethyl, 4-nitrophenethyl, and 2,2,2-trichloroethyl.
8. The liquid-phase process as defined in any one of the preceding statements, wherein PG is selected from the group consisting of cyanoethyl, 2-chlorophenyl, 4-chlorophenyl, 2,2,2-trichloroethyl and methyl.
9. The liquid-phase process as defined in any one of the preceding statements, wherein PG is cyanoethyl.
10. The liquid-phase process as defined in any one of the preceding statements, wherein step c) comprises isolating the compound of formula II from one or more reactants used in step b).
11. The liquid-phase process as defined in any one of the preceding statements, wherein during step c), the compound of formula II is collected in the retentate.
12. The liquid-phase process as defined in any one of the preceding statements, wherein step c) is conducted in an organic solvent or a mixture of organic solvents.
13. The liquid-phase process as defined in any one of the preceding statements, wherein the membrane is a crosslinked polybenzimidazole membrane.
14. The liquid-phase process as defined in any one of the preceding statements, wherein step b) comprises reacting the compound of formula I with a compound of formula A:

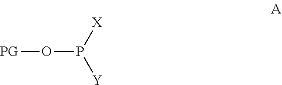

in which PG and X are as defined in any one of the preceding statements, and Y is halo or has any of those definitions outlined in any one of the preceding statements for X;
wherein when Y is not halo, step b) is conducted in the presence of a first phosphoramidite activator.
15. The liquid-phase process as defined in statement 14, wherein Y is Cl or —N(Pr)₂.
16. The liquid-phase process as statements in 14, wherein the compound of formula A is:

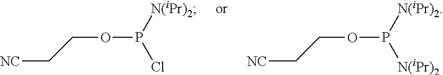

17. The liquid-phase process as defined in statement 14, 15 or 16, wherein the first phosphoramidite activator is capable of reacting with the compound of formula A to form a compound of formula A′:

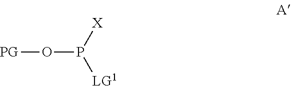

wherein X and PG are as defined in any one of the preceding statements, and
LG¹ is a leaving group;
and wherein the compound of formula A′ is capable of reacting with the compound of formula I to formula the compound of formula II.
18. The liquid-phase process as defined in statement 17, wherein LG¹ is aza-heteroaryl (e.g., imidazolyl, pyridyl or tetrazolyl).
19. The liquid-phase process as defined in any one of statements 14 to 18, wherein the first phosphoramidite activator is:

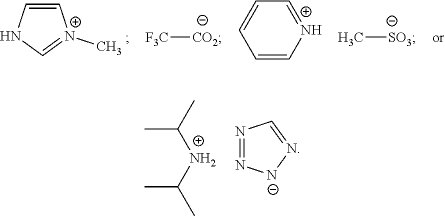

20. The liquid-phase process as defined in any one of statements 14 to 19, wherein the first phosphoramidite activator is:

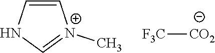

21. The liquid-phase process as defined in any one of the preceding statements, wherein step e) is conducted in the presence of a second phosphoramidite activator.
22. The liquid-phase process as defined in statement 21, wherein the second phosphoramidite activator is capable of reacting with the compound of formula II to form a compound of formula II′:

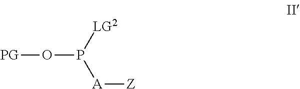

in which Z, A and PG are as defined in any preceding statement;
LG² is a leaving group;
and wherein the compound of formula II′ is capable of reacting with the compound of formula III to form the compound of formula IV.
23. The liquid-phase process as defined in statement 22, wherein LG² is aza-heteroaryl.
24. The liquid-phase process as defined in statement 22, wherein LG² is imidazolyl or tetrazolyl.
25. The liquid-phase process as defined in statement 22, wherein LG² is tetrazole, 5-(ethylthio)-1H-tetrazole, 4,5-dicyanoimidazole or 5-(benzylthio)-1H-tetrazole.
26. The liquid-phase process as defined in statement 21, wherein the second phosphoramidite activator is an aza-heterocycle.
27. The liquid-phase process as defined in statement 21, wherein the second phosphoramidite activator is tetrazole, 5-(ethylthio)-1H-tetrazole, 4,5-dicyanoimidazole or 5-(benzylthio)-1H-tetrazole.
28. The liquid-phase process as defined in any one of the preceding statements, wherein step e) is conducted under anhydrous conditions.
29. The liquid-phase process as defined in any one of the preceding statements, wherein: (i) when A is a nucleoside, Z is attached at the 3′ terminal of A, with T^{A} disposed at the 5′ terminal of A, or vice versa; and (ii) when A is an oligonucleotide, Z is attached at the 3′ terminal of one distal nucleoside of A, with T^{A} disposed at the 5′ terminal of the other distal nucleoside of A, or vice versa.
30. The liquid-phase process as defined in any one of the preceding statements, wherein T^{A} is disposed at the 5′ terminal of the nucleoside or oligonucleotide, A, and Z is disposed at the 3′ terminal of the nucleoside or oligonucleotide, A.
31. The liquid-phase process as defined in any one of the preceding statements, wherein the compound of formula I has a structure according to formula Ia:

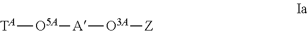

wherein
—O^{5A}-A′-O^{3A}— is a nucleoside or an oligonucleotide, in which O^{5A} is the oxygen located at the terminal 5′ carbon and O^{3A} is the oxygen located at the terminal 3′ carbon;
T^{A} is hydrogen; and
Z is a soluble synthesis support.
32. The liquid-phase process as defined in any one of the preceding statements, wherein when the compound of formula I is a compound of formula Ia, the compound of formula II may be a compound of formula IIa:

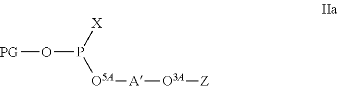

in which PG, X, —O^{5A}-A′-O^{3A}— and Z are as defined in any preceding statement.
33. The liquid-phase process as defined in any one of the preceding statements, wherein: (i) when B is a nucleoside, T^{B1} is disposed at the 3′ terminal of B, with T^{PG} disposed at the 5′ terminal of B, or vice versa; and (ii) when B is an oligonucleotide, T^{B1} is disposed at the 3′ terminal of one distal nucleoside of B, with T^{PG} disposed at the 5′ terminal of the other distal nucleoside of B, or vice versa.
34. The liquid-phase process as defined in any one of the preceding statements, wherein T^{B1} is disposed at the 3′ terminal of the nucleoside or oligonucleotide, B, and T^{PG} is disposed at the 5′ terminal of the nucleoside or oligonucleotide, B.
35. The liquid-phase process as defined in any one of the preceding statements, wherein T^{PG} is a protecting group bound to the 5′ oxygen of B.
36. The liquid-phase process as defined in any one of the preceding statements, wherein T^{PG} is an acid-labile protecting group, non-limiting examples of which include dimethoxytrityl (Dmtr/DMT), tert-butyl (tBu), tert-butyl oxycarbonyl (Boc), mono-methoxytriphenyl (Mmtr), triphenyl methyl (Tr), pentamethyl dihydrobenzofuran sufonyl (Pbf), Tetrahydropyranyl (Thp), tetrahydrofuranyl (Thf), para-methoxybenzyl (Pmb) and 2,4-dimethoxybenzyl
37. The liquid-phase process as defined in any one of the preceding statements, wherein T^{PG} is dimethoxytrityl bound to the 5′ oxygen of B.
38. The liquid-phase process as defined in any one of the preceding statements, wherein the compound of formula III has a structure according to formula Illa:

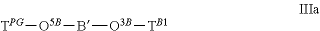

wherein
—O^{5B}—B′—O^{3B}— is a nucleoside or an oligonucleotide, in which O^{5B} is the oxygen located at the terminal 5′ carbon and O^{3B} is the oxygen located at the terminal 3′ carbon;
T^{B1} is hydrogen; and
T^{PG} is a protecting group (e.g., DMT).
39. The liquid-phase process as defined in any one of the preceding statements, wherein when the compound of formula I is a compound of formula Ia, and the compound of formula III is a compound of formula Illa, the compound of formula IV is a compound of formula IVa:

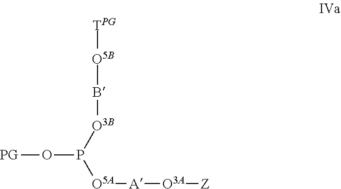

in which PG, —OBA-A′-O^{3A}, Z, —O^{5B}—B′—O^{3B}— and T^{PG} are as defined in any preceding statement.
40. The liquid-phase process as defined in any one of the preceding statements, wherein Z is a polymeric soluble synthesis support.
41 The liquid-phase process as defined in any one of the preceding statements, wherein Z has a molecular weight (M_{w}) of 500-50,000 Da.
42. The liquid-phase process as defined in any one of statements 1-40, wherein the Z is a compound of formula B:

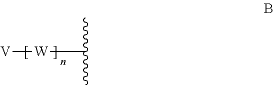

in which
V is an organic branch point,
W is a polymeric chain,
[symbol] represents a point of attachment to A, and
n is 1-8 (e.g., 1-6).
43. The liquid-phase process as defined in statement 42, wherein n is 3-4,
44. The liquid-phase process as defined in statement 42 or 43, wherein V is aliphatic or aromatic.
45. The liquid-phase process as defined in statement 42, 43 or 44, wherein each W has a molecular weight (M_{w}) of 500-20,000 Da (e.g., 2000-15,000 Da or 8000-12,000 Da)
46. The liquid-phase process as defined in statement 42, wherein Z is a compound of formula B having the structure:

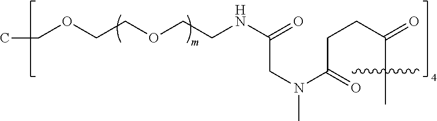

in which [symbol] represents a point of direct attachment to A (e.g., to the 3′ oxygen of A) and m is 100-500 (e.g., 200-300).
47. The liquid-phase process as defined in any one of the preceding statements, further comprising an oxidation step, in which one of more P(III) phosphite triester linkages is converted to a P(V) phosphotriester linkage (e.g., by oxidation or sulfur transfer).
48. The liquid-phase process as defined in statement 47, wherein the product resulting from the oxidation step is isolated by membrane filtration.
49. The liquid-phase process as defined in any one of the preceding statements, further comprising a T^{PG} deprotection step, in which the oligonucleotide is contacted with a deprotecting agent to form a compound of formula V:

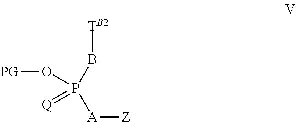

in which
Z and A are as defined in relation to formula I;
PG is as defined in relation to formula II;
Q is selected from absent, O or S; and
T^{B2} is a reactive terminal of B (e.g., a 5′ terminal).
50. The liquid-phase process as defined in statement 49, further comprising the step of isolating the compound of formula V by membrane filtration.
51. The liquid-phase process as defined in any one of statements 47 to 50, wherein the oxidation step is performed after step e) and the T^{PG} deprotection step is performed after the oxidation step.
52. The liquid-phase process as defined in any one of the preceding statements, further comprising the steps of:
(i) providing a growing oligonucleotide, wherein the growing oligonucleotide is:
   A) the compound of formula V, or
   B) the compound of formula V, to which has been coupled one or more additional nucleosides, wherein the nucleoside most distant from Z has a reactive terminal;
(ii) modifying the growing oligonucleotide to form a compound of formula VI:

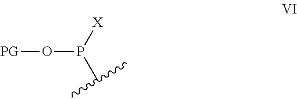

in which [symbol] represents a bond to the growing oligonucleotide (e.g., at the reactive terminal), and PG and X are as defined in relation to formula II;
(iii) isolating the compound of formula VI by membrane filtration;
(iv) providing a nucleoside or oligonucleotide building block, having a reactive terminal and a protected terminal;
(v) reacting the isolated compound of formula VI with the reactive terminal of the nucleoside or oligonucleotide building block to form a chain-extended oligonucleotide, the chain-extended oligonucleotide having a protected terminal;
(vi) isolating the chain-extended oligonucleotide by membrane filtration;
(vii) deprotecting the protected terminal of the isolated chain-extended oligonucleotide, and optionally repeating the sequence of steps (i) to (vii).
53. The liquid-phase process as defined in statement 52, further comprising the step of converting one of more P(III) phosphite triester linkages to a P(V) phosphotriester linkage (e.g., by oxidation or sulfur transfer), optionally wherein the step is performed between steps (v) and (vi).
54. The liquid-phase process as defined in any one of the preceding statements, further comprising the step of removing the protecting group PG from one of more P(V) phosphotriester linkages.
55. The liquid-phase process as defined in any one of the preceding statements, further comprising the step of cleaving the oligonucleotide from Z.
56. The liquid-phase process as defined in any one of the preceding statements, wherein the process is conducted in acetonitrile, or in a solvent mixture comprising acetonitrile.
57. The liquid-phase process as defined in any one of the preceding statements, wherein the process is conducted in a closed-loop reactor.

### EXAMPLES

One or more examples of the invention will now be described, for the purpose of illustration only, with reference to the accompanying figures:

FIG. 1. Oligonucleotide synthesis cycle by forward phosphoramidite coupling approach. Coupling between phosphoramidite 4 and the 5′-OH is initiated by one of a wide range of acidic activators. Equally, there exists a wide range of reagents to convert the P(III) phosphite tri-ester to a P(V) phospho (X=O or S) tri-ester. In SPOS a capping reagent is used to block any residual unreacted 5′-OH (commonly as an acetate ester), but this step can be omitted in LPOS. In SPOS washing of the support is used to drive detritylation to completion, but in LPOS a cation scavenger may be used. In membrane assisted phosphoramidite LPOS, points in the cycle for organic solvent nanofiltration (OSN) are indicated.

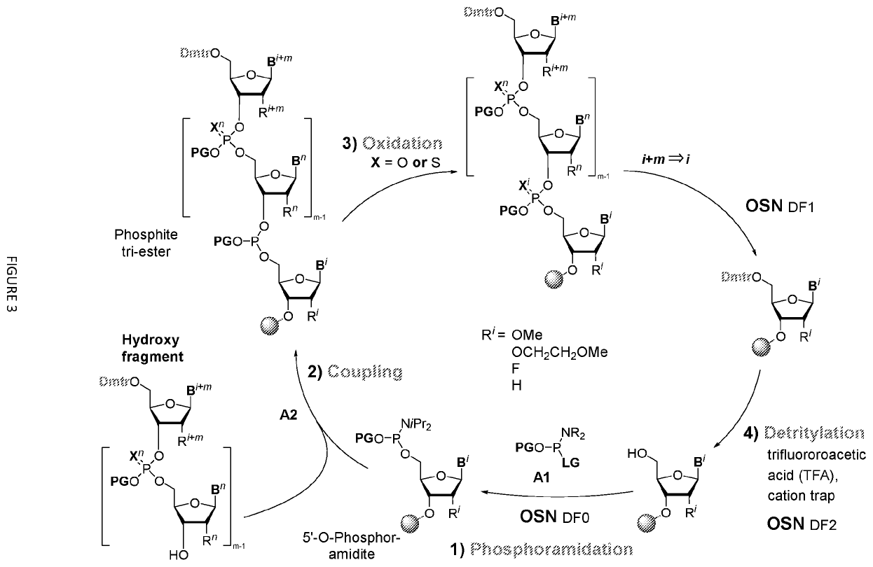

FIG. 2. Reversed phosphoramidite coupling approach to LPOS. The oxidation and detritylation steps are the same as for the forward phosphoramidite approach to oligo synthesis (FIG. 1). The initial phosphoramidation reaction used a weakly acidic phosphoramidite activator, A1. Once excess bis-amidite has been washed away, the coupling reaction, triggered by a more acidic phosphoramidite activator, A2, is typically performed under anhydrous conditions to suppress capping of the growing oligo chain, initially as an H-phosphonate. Non-limiting examples of activators, phosphate tri-ester protecting groups (PG) and leaving groups (LG) are shown.

FIG. 3. Reversed phosphoramidite coupling of fragments.

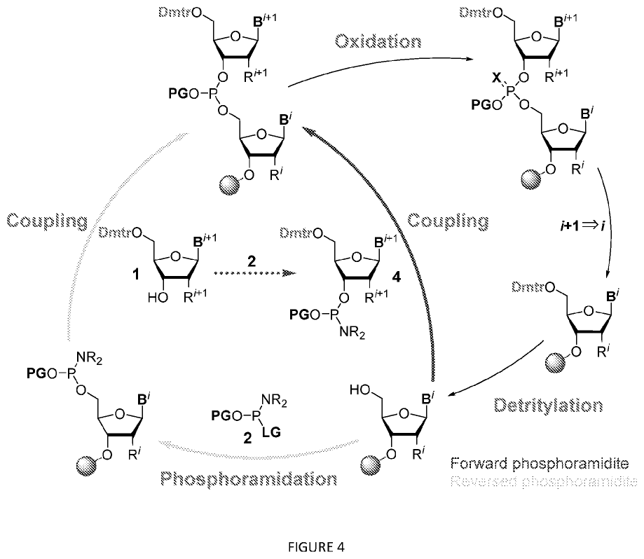

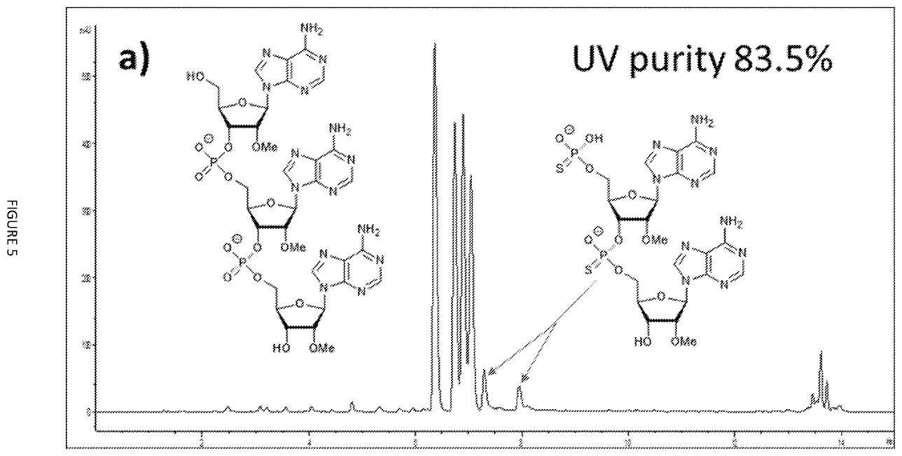

FIG. 4. Combination of reversed and forward phosphoramidite approaches in the oligo chain extension cycle.

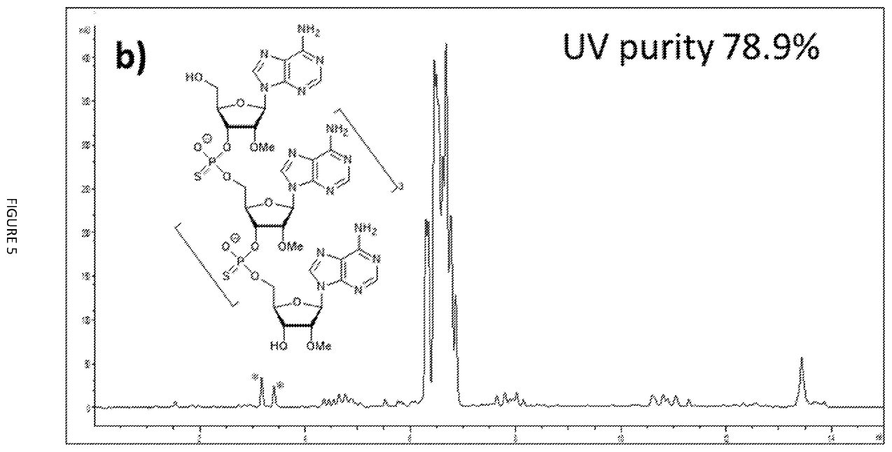

FIG. 5 Ion-pair reversed phase UV chromatograms of homo-mA sequences during synthesis: a) mA₃ trimer-note that the principal contaminant is the 5′-phosphorothioate dimer, which derives from S-transfer to an H-phosphonate, therefore significant water ingress occurred; b) mA₅ pentamer-starred peaks are the 5′-phosphorothioate dimer in a), demonstrating that the error is capped. At pentamer phosphorothioate capping accounts for 13.4% total impurities; n-x (mainly x=1), 3.1%; PO, 0.3%; +Cne, 0.3%.

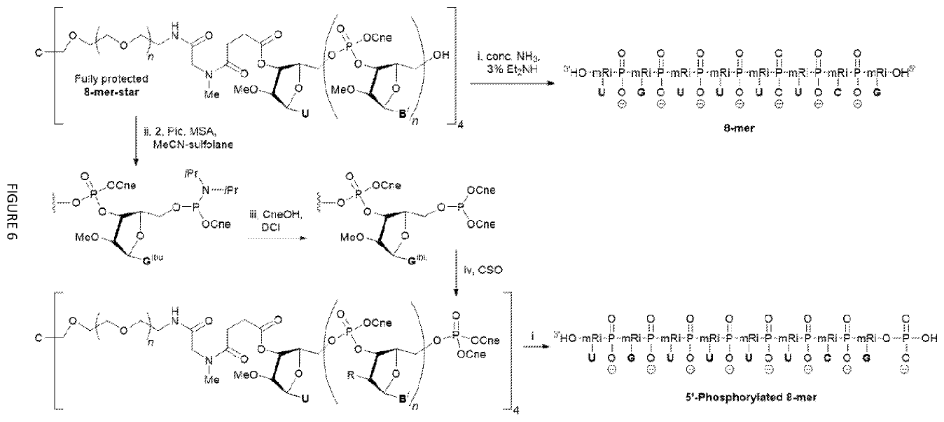

FIG. 6. Installation of a 5′-phosphate by reversed phosphitylation according to Example 2.

A typical synthetic cycle in the synthesiser consists of three steps (see FIG. 2): 1) phosphoramidation, then membrane diafiltration (DF) in anhydrous organic solvent, a.k.a. organic solvent nanofiltration (OSN; DF0, 4 DV) to remove the excess bis-amidite agent; 2) coupling, and 3) oxidation or sulfur transfer, followed by OSN (DF1, 4 diavolumesd or DV) to remove excess nucleoside, activator and oxidant—this DF can be omitted; and finally, 4) detritylation, followed by purification of the crude oligo via OSN (DF2, 6 DV). The last DF removes any residual detritylated nucleoside that would otherwise participate in the next chain extension cycle.

Below representative loading and chain extension cycles in a membrane synthesiser are described. Note: Because membrane synthesisers are sensitive to fouling of the membrane by fine particulates, all solutions injected into the synthesiser were filtered through a PTFE membrane (0.2 μm, or tighter).

### Example 1: a Homo-Pentamer, all Phosphorothioate

Loading of 5-Dmtr-mA^{Bz} 3′-Succinate onto 10 kDa PEG-Star and Charging of the Nanostar Synthesizer

PEG-10k (Sar-H)₄ (11 g, 1.068 mmol) and 5′-Dmtr-mA^{Bz} 3′-succinate, triethylammonium salt (6.2 g, 8 mmol, 8 eq.) were placed in round-bottomed flask. The starting materials were dissolved MeCN (33 mL), after which DCC (1.65 g, 8 mmol, 8 eq.) and HOBt hydrate (1.081 g, 8 eq., 8 mmol) were added. The amidation reaction was monitored by LC-MS; typically, the reaction is stirred overnight to ensure completion. The crude solution was then filtered through a PTFE filter (0.1 μm, Sterlitech Pressurised Filtration Holder) into a round bottom flask. The filtrate was transferred to the synthesiser feed tank via a port using a plastic funnel, washing out the flask with further MeCN. After a sample had been taken from the fully mixed Nanostar synthesiser to determine the residual Dmtr-succinate concentration at the start of DF, MeCN was then permeated (1 L=4DV, DF1). At the end of DF1 another sample was analysed by LC-MS to measure the fractional drop in the concentration of succinate, and the rejection of the trityl nucleoside-star.

### Detritylation Reaction

A solution of cation trap (4 eq./arm, 16 in total; dodecanthiol can be used for this purpose) in MeCN (10 mL) was injected into the feed tank via the injection port using a syringe fitted with disposable PTFE filter disc (0.2 μm). Dmtr deprotection was then initiated by injecting TFA (12.5 mL=5 vol %). The detritylation reaction was monitored by LC-MS and after 10 min no partially deprotected species could be detected. After 20 min the reaction was quenched with pyridine (25 mL) and a sample was taken to determine the intermediate concentration of detritylated succinate. Dry MeCN (1.5 L=6DV, <20 ppm water) was permeated (DF2), to remove residual 5′-hydroxy-mA^{Bz} 3′-succinate and other detritylation debris. At the end of DF2 a final sample was analysed by LC-MS to verify complete removal of building block debris and the high rejection of the nucleoside-star. Only proceed to the next step if the combined drop in building block concentration over DF1 and DF2 is >99%, or else continue DF2 until this condition is satisfied.

### Phosphoramidation Reaction

Before proceeding with the phosphitylation, the reactor is dried; if it is left unpressurised and not diafiltering, ambient moisture will slowly make ingress into the reactor. The moisture content of the reactor is inferred by measuring the moisture content of the permeate at the end of DF2 using a Karl-Fischer coulometric titrator. Values of <40 ppm water are acceptable to proceed to the phosphoramidation step, otherwise further DF2 is recommend until this value is achieved.

1-Methyl Imidazole (NMI, 0.947 g, 11.53 mmol, 2.7 eq./arm) was placed in a 50 mL round-bottomed flask and dissolved in anhydrous MeCN (15 mL, <20 ppm water) under an inert atmosphere. To this solution was added TFA (0.817 mL, MW=114.02, d=1.49 g/mL, 10.68 mmol, 2.5 eq./arm) followed by the addition of 500 mg of MgSO₄ as drying. The solution of activator was then stirred for 10 min at 25 deg. C.

Bis-amidite N,N,N′,N′-tetraisopropyl phosphorodiamidite (2, 4.07 mL, 12.81 mmol, 3 eq./arm) was injected into the synthesizer and allowed to circulate for 2 minutes. The pre-formed NMI·TFA activator solution was then injected into the synthesizer via a PTFE filter (0.2 micron).

The phosphoramidation reaction was monitored by LC: a sample from the synthesiser (0.1 mL) is added to a mixed solution of excess dicyanoimidazole and cyanoethanol (0.2 mL), and after 2 minutes the solution is added to excess xanthane hydride in pyridine (0.2 mL); this converts reactive oligo-star 5′-O-phosphoramidite to a stable dicyanoethanol phosphorothioate tri-ester. The processed sample is then diluted with MeCN (0.6 mL) and analysed by LC: Waters Acquity UPLC, C18 column; solvent A, 50 mM sodium acetate in water; solvent B, MeCN-MeOH 4:1; temperature 60 deg. C., 20-100% solvent B over 6 min. After 5 min all the phosphoramidation intermediates are consumed, and after 20 min diafiltration was commenced. After anhydrous MeCN (<10 ppm water; 1 L=4DV) has been permeated (DF0), the oligo-star 5′-O-phosphoramidite is ready to be coupled with the next nucleoside. The water content of the membrane reactor, determined from sampling of the permeate and KF coulometric analysis, should be <40 ppm or less to proceed to the coupling step.

### Coupling Reaction

5′-Dmtr-mA^{Bz} nucleoside (4.4 g, 6.4 mmol, 1.5 eq./arm) and 4,5-dicyanoimidazole (DCI, 2.018 g, MW=118.1 Da, 17 mmol, 4 eq./arm) were each placed in a separate round bottom flasks. The building block was co-evaporated from MeCN (<15 ppm water, 3×100 mL), re-dissolved in MeCN (20 mL) and injected into the synthesizer via a PTFE filter (0.2 micron). The DCI was dissolved in MeCN (20 mL) and also injected into the synthesizer via a PTFE filter (0.2 micron). The reaction was monitored by LC: After 2 minutes the coupling intermediates were consumed, but circulation was continued in the synthesizer for 25 minutes to ensure complete reaction. 3-Phenyl 1,2,4-dithiazoline-5-one (POS, 3.33 g, 17 mmol, 4 eq./arm) dissolved in MeCN (80 mL) was then added to the synthesiser to obtain the chain extended phosphorothioate tri-ester. The crude dimer-star was purified by diafiltration with MeCN (1 L=4 DV).

The synthetic cycle described above was repeated in the same fashion (changing the building block as required) to elongate the oligo chain up to the desired length.

### Global Deprotection

During the synthesis, oligo-stars were periodically analysed by global deprotection of a small sample and the purities of the crude oligos were estimated by analytical ion-pair reversed phase UHPLC chromatography. The sample preparation method is described below:

A sample of crude HO-oligo-star solution (3 mL, ca. 0.048 mmol oligo) was withdrawn from the synthesizer and placed in an ACE pressure tube in concentrated aq. ammonia (~3 mL). To this solution diethylamine (0.1 mL) was added and the tube was sealed and heated at 35° C. overnight. The next day the solution was transferred to a round bottom flask and evaporated. The aqueous residue was co-evaporated from MeCN three times, and the residue was finally triturated with MeCN. The suspension was transferred to a Falcon tube (~30 mL) and centrifuged (5 min, 6000 rpm). The supernatant was removed, and the precipitate washed and centrifuged twice more with further MeCN. The precipitate was then analysed on an Agilent UHPLC by IP-RP.

### Example 2: Installing a 5′-Phosphate by Reversed Phosphitylation

### 5′-Phosphoramidation

Having regard to FIG. 6, 3-Methyl picoline (0.255 mL, 2.6 mmol, 2.62 eq./arm) was dissolved in anhydrous MeCN (5 mL, <20 ppm water) under an inert atmosphere, and to this solution was added methane sulfonic acid (MSA, 0.162 mL, 2.5 mmol, 2.5 eq./arm). Molecular sieves (3 Å, 5 g bag) were added to the solution which was stirred overnight at 25 deg. C. to provide an anhydrous solution of Pic.MSA activator.

The next day PEG-10k (Sar-Suc-mU-mGIbu_mU-mU-mU-mU-mCAc-mGIbu) 4 (3.15 g, 0.25 mmol) was dissolved in anhydrous MeCN-sulfolane (80:20 v/v, 5 mL) and transferred by syringe to the feed tank a mini-synthesizer (system volume 50 mL) through a septum port, washing out the flask with further MeCN-sulfolane. Diafiltration with anhydrous MeCN: sulfolane (80:20 v/v, <20 ppm water) was continued until the moisture content of the permeate, determined using a Karl-Fischer coulometric titrator, was <30 ppm water.

N,N,N′,N′-Tetraisopropyl phosphorodiamidite (0.953 mL, 3 mmol, 3 eq./arm) was then injected into the synthesizer and allowed to circulate for 2 minutes. Next, the pre-formed Pic.MSA activator solution was injected into the synthesizer via a PTFE filter (0.2 micron). The phosphoramidation reaction was monitored by LC: A sample from the synthesiser (0.1 mL) was added to a solution containing dicyanoimidazole (100 mg) and cyanoethanol (0.2 mL), and after 2 minutes the solution was transferred another sample tube containing xanthane hydride (100 mg) in pyridine (0.2 mL); this converts reactive oligo-star 5′-O-phosphoramidite to a stable dicyanoethanol phosphorothioate tri-ester. The processed sample was then diluted with MeCN (0.6 mL) and analysed by LC: Waters Acquity UPLC, C18 column; solvent A, 50 mM sodium acetate in water; solvent B, MeCN-MeOH 4:1; temperature 60 deg. C., 20-100% solvent B over 6 min. After 30 min all the phosphoramidation intermediates were consumed.

### Conversion to 5′-Phosphate and Global Deprotection

Ethylene cyanohydrin (410 μL, 6 mmol, 6 eq./arm) was injected into the synthesizer, followed by dicyanoimidazole (DCI, 0.710 g, 6 mmol, 6 eq./arm) dissolved in MeCN (5 mL). After 20 minutes, camphorsulfonyl oxaziridine (CSO, 0.51 g, 2.25 mmol, 9 eq./arm) dissolved in MeCN (10 mL) was added to the synthesiser to convert the chain termini to 5′-dicyanoethyl phosphotriesters. The crude phosphorylated 8-mer-star was purified by diafiltration with MeCN-sulfolane 80:20 v/v (200 mL=4 DV).

A sample of the purified 5-phosphorylated 8-mer-star solution (2 mL, ca. 0.01 mmol oligo) was withdrawn from the synthesizer and placed in an ACE pressure tube in concentrated aq. ammonia (~2 mL). Diethylamine (120 L) was added, the tube was sealed and heated at 35° C. overnight. The next day the solution was transferred to a round bottom flask and evaporated. The aqueous residue was co-evaporated from MeCN three times, and the residue was finally triturated with MeCN. The suspension was transferred to a Falcon tube (~30 mL) and centrifuged (5 min, 6000 rpm). The supernatant was removed, and the precipitate washed and centrifuged twice more with further MeCN. The precipitate was then analysed on an Agilent UHPLC by analytical ion-pair reversed phase (IP-RP) UHPLC chromatography: Mobile phase A Optima water-MeOH 9:1, 2 UM EDTA, 60 mM HFIP, 6 mM HA; mobile phase B MeCN:MeOH 1:1, Acquity Premier Peptide BEH C18 Column, 300 Å, 1.7 μm, 2.1×150 mm, column temperature 65 deg. C., detector 260 nm, sample temperature 10 deg. C., method 5-25% B, 15 min run time, 0.4 mL/min, 2 μL injection volume. The desired 5′-phosphorylated 8-mer eluted at 10.46 min, compared to the unphosphorylated 8-mer at 8.10 min.

While specific embodiments of the invention have been described herein for the purpose of reference and illustration, various modifications will be apparent to a person skilled in the art without departing from the scope of the invention as defined by the appended claims.

### BIBLIOGRAPHY

U.S. Pat. No. 8,664,357

U.S. Pat. No. 9,127,123

U.S. Pat. No. 10,239,996

EP3347402

EP0813539 B1

EP1612213 B1

WO 2016/188835 A1

U.S. Pat. No. 7,960,542 B2
P. R. J. Gaffney, J. F. Kim, I. B. Valtcheva, G. D. Williams, M. S. Anson, A. M. Buswell, A. G. Livingston, Liquid-Phase Synthesis of 2′-Methyl-RNA on a Homostar Support through Organic-Solvent Nanofiltration, Chem. Eur. J., 2015, 21, 9535-9543
J. F. Kim, P. R. J. Gaffney, I. B. Valtcheva, G. Williams, A. M. Buswell, M. S. Anson, A. G. Livingston, Organic Solvent Nanofiltration (OSN): A New Technology Platform for Liquid-Phase Oligonucleotide Synthesis (LPOS), Org. Process Res. Dev. 2016, 20, 1439-1452
So Su, Peeva L. G., Tate E. W., Leatherbarrow R. J., Livingston A. G. “Organic Solvent Nanofiltration-A New Paradigm in Peptide Synthesis” Org. Process. Res. & Dev. 14 (2010) pp. 1313-132
Yeo J, Peeva L, Chung S, Gaffney P, Kim D, Luciani C, Tsukanov S, Siebert K, Kopach M, Albericio F, Livingston A “Liquid Phase Peptide Synthesis by One Pot Nanostar Sieving (PEPSTAR)”; Angew. Chem. Int. Ed. 2021, 60, pp 7786-7795
Dong R., Liu R., Gaffney P. R. J., Schaepertoens M., Marchetti P., Williams C. M., Chen R. and Livingston A. G. “Sequence-defined multifunctional polyethers via liquid-phase synthesis with molecular sieving” Nature Chemistry (2019) 11 pp. 136-145
B. I. Andrews, F. D. Anita, S. B. Brueggemeier, L. J. Diorazio, S. G. Koenig, M. E. Kopach, H. Lee, M. Olbrich, A. L Watson, Sustainability challenges and opportunities in oligonucleotide manufacturing, J. Org. Chem., 2021, vol. 86, p. 49-61.
A. Scozzari, Oligonucleotides for large market indications, what needs to happen or be considered? TKS Webinar Series-Innovations in Oligonucleotide Therapeutics Manufacturing, 2021.
E. Legorburu, C. B. Reese, Q. Song, Conversion of uridine into 2′-O-(2-methoxyethyl) uridine and 2′-O-(2-methoxyethyl) cytidine, Tetrahedron, 1995, v55, p5635-5640.
P. Liu, A. Sharon, C. K. Chu, Fluorinated nucleosides; Synthesis and biological implication, J. Fluorine Chem., 2008, v129, p743-766.
J. Nielsen, M. Taagaard, J. E. Marugg, J. H. van Boom, O. Dahl, Application of 2-cyanoethyl N,N,N′,N,-tetraisopropylphosphorodiamidite for in situ preparation of deoxyribonuceoside phosphoramidites and their use in polymer-supported synthesis of oligonucleotides, Nucl. Acids Res., 1986, vol 14, p7391-7403.
D. S. Pedersen, C. Rosenbohm, T. Koch, Synthesis, 2002, p802-808.
Y. S. Sanghvi, Z. Guo, H. M. Pfundheller, A. Converso, Improved Process for the Preparation of Nucleosidic Phosphoramidites Using a Safer and Cheaper Activator, Org. Proc. Res. Dev., 2000, v4, p175-181
C. Xie, M. I A. Staszak, J. T. Quatroche, C. D. Sturgill, V. V. Khau, M. J. Martinelli, Nucleosidic Phosphoramidite Synthesis via Phosphitylation: Activator Selection and Process Development, Org. Proc. Res. Dev., 2005, v9, p730-737.
A. F. Sandahl PhD thesis, Method developments in solid-phase chemistry: Easing access to synthetic oligonucleotides and oligo (disulfides), Aarhus University, Denmark, 2020.

1. A liquid-phase process for forming an oligonucleotide, the process comprising the steps of:
a) providing a compound of formula I: 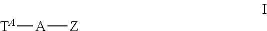
wherein A is a nucleoside or an oligonucleotide, T^{A} is a reactive terminal of the nucleoside or oligonucleotide, and Z is a soluble synthesis support to which the nucleoside or oligonucleotide is attached;
b) modifying the compound of formula I to form a compound of formula II: 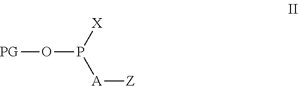
in which A and Z are as defined in respect of formula I, X is a tertiary amino group and PG is a protecting group;
c) isolating the compound of formula II by membrane filtration;
d) providing a compound of formula III: 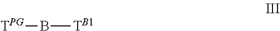
wherein B is a nucleoside or an oligonucleotide, T^{B1} is a reactive terminal of the nucleoside or oligonucleotide, and T^{PG} is a protected terminal of the nucleoside or oligonucleotide;
e) reacting the compound of formula II with the compound of formula III to form a compound of formula IV: 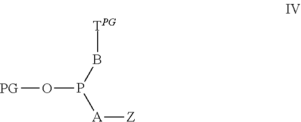
in which A and Z are as defined in respect of formula I, PG is as defined in respect of formula II, and B and T^{PG} are as defined in respect of formula III.

2. The liquid-phase process as claimed in claim 1, wherein X is a group —NR₂, in which each R is independently selected from (1-6C)alkyl, or both R groups are linked such that when taken in combination with the nitrogen atom to which they are attached, they collectively form a 5- to 7-membered heterocycle.

3. (canceled)

4. The liquid-phase process as claimed in claim 1, wherein PG is selected from the group consisting of cyanoethyl, 2-chlorophenyl, 4-chlorophenyl, 2,2,2-trichloroethyl and methyl.

5. The liquid-phase process as claimed in claim 1, wherein step c) comprises isolating the compound of formula II from one or more reactants used in step b).

6. The liquid-phase process as claimed in claim 1, wherein step b) comprises reacting the compound of formula I with a compound of formula A: 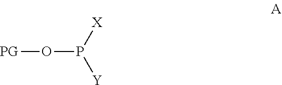
in which PG and X are as defined in claim 1, and Y is halo or is as defined in claim 1 for X;
wherein when Y is not halo, step b) is conducted in the presence of a first phosphoramidite activator.

7. The liquid-phase process as claimed in claim 6, wherein the first phosphoramidite activator is capable of reacting with the compound of formula A to form a compound of formula A′: 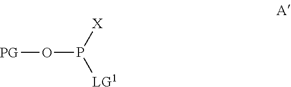
wherein X and PG are as defined in claim 1, and LG¹ is a leaving group;
and wherein the compound of formula A′ is capable of reacting with the compound of formula I to formula the compound of formula II.

8. (canceled)

9. The liquid-phase process as claimed in claim 1, wherein
(a) T^{A} is disposed at the 5′ terminal of the nucleoside or oligonucleotide, A, and Z is disposed at the 3′ terminal of the nucleoside or oligonucleotide, A; and/or
(b) T^{B1} is disposed at the 3′ terminal of the nucleoside or oligonucleotide, B, and T^{PG} is disposed at the 5′ terminal of the nucleoside or oligonucleotide, B.

10. The liquid-phase process as claimed in claim 1, wherein the compound of formula I has a structure according to formula Ia: 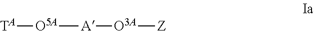
wherein
—O^{5A}-A′-O^{3A}— is a nucleoside or an oligonucleotide, in which O^{5A} is the oxygen located at the terminal 5′ carbon and O^{3A} is the oxygen located at the terminal 3′ carbon;
T^{A} is hydrogen; and
Z is a soluble synthesis support.

11. The liquid-phase process as claimed in claim 1, wherein T^{PG} is a protecting group bound to the 5′ oxygen of B; optionally wherein T^{PG} is an acid-labile protecting group.

12. (canceled)

13. The liquid-phase process as claimed in claim 1, wherein the compound of formula III has a structure according to formula IIIa: 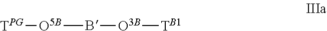
wherein
—O^{5B}—B′—O^{3B}— is a nucleoside or an oligonucleotide, in which O^{5B} is the oxygen located at the terminal 5′ carbon and O^{3B} is the oxygen located at the terminal 3′ carbon;
T^{B1} is hydrogen; and
T^{PG} is a protecting group (e.g., DMT).

14. The liquid-phase process as claimed in claim 1, wherein step e) is conducted in the presence of a second phosphoramidite activator.

15. The liquid-phase process as claimed in claim 14, wherein the second phosphoramidite activator is capable of reacting with the compound of formula II to form a compound of formula II′: 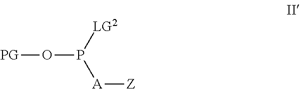
in which Z, A and PG are as defined in claim 1;
LG² is a leaving group;
and wherein the compound of formula II′ is capable of reacting with the compound of formula III to form the compound of formula IV.

16. (canceled)

17. The liquid-phase process as claimed in claim 1, wherein Z is a polymeric soluble synthesis support, optionally wherein Z has a molecular weight (M_{w}) of 500-50,000 Da.

18. The liquid-phase process as claimed in claim 1, wherein the Z is a compound of formula B: 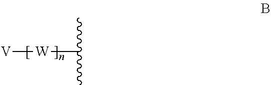
in which
V is an organic branch point,
W is a polymeric chain, [symbol]
represents a point of attachment to A, and
n is 1-8.

19. (canceled)

20. The liquid-phase process as claimed in claim 1, further comprising an oxidation step, in which one of more P(III) phosphite triester linkages is converted to a P(V) phosphotriester linkage (e.g., by oxidation or sulfur transfer);
and wherein the product resulting from the oxidation step is isolated by membrane filtration.

21. The liquid-phase process as claimed in claim 1, further comprising a T^{PG} deprotection step, in which the oligonucleotide is contacted with a deprotecting agent to form a compound of formula V: 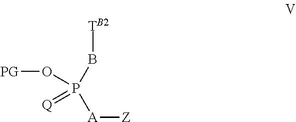
in which
Z and A are as defined in relation to formula I;
PG is as defined in relation to formula II;
Q is selected from absent, O or S; and
T^{B2} is a reactive terminal of B (e.g., a 5′ terminal); and
further comprising the step of isolating the compound of formula V by membrane filtration.

22. The liquid-phase process as claimed in claim 20, wherein the oxidation step is performed after step e) and the T^{PG} deprotection step is performed after the oxidation step.

23. The liquid-phase process as claimed in claim 1, further comprising the steps of:
(i) providing a growing oligonucleotide, wherein the growing oligonucleotide is:
A) the compound of formula V, or
B) the compound of formula V, to which has been coupled one or more additional nucleosides, wherein the nucleoside most distant from Z has a reactive terminal;
(ii) modifying the growing oligonucleotide to form a compound of formula VI: 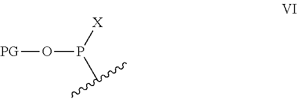
in which [symbol] represents a bond to the growing oligonucleotide (e.g., at the reactive terminal), and PG and X are as defined in relation to formula II;
(iii) isolating the compound of formula VI by membrane filtration;
(iv) providing a nucleoside or oligonucleotide building block, having a reactive terminal and a protected terminal;
(v) reacting the isolated compound of formula VI with the reactive terminal of the nucleoside or oligonucleotide building block to form a chain-extended oligonucleotide, the chain-extended oligonucleotide having a protected terminal;
(vi) isolating the chain-extended oligonucleotide by membrane filtration;
(vii) deprotecting the protected terminal of the isolated chain-extended oligonucleotide, and optionally repeating the sequence of steps (i) to (vii).

24. The liquid-phase process as claimed in claim 23, further comprising the step of converting one of more P(III) phosphite triester linkages to a P(V) phosphotriester linkage (e.g., by oxidation or sulfur transfer), optionally wherein the step is performed between steps (v) and (vi).

25. The liquid-phase process as claimed in claim 1, further comprising
(a) the step of removing the protecting group PG from one of more P(V) phosphotriester linkages; and/or
(b) the step of cleaving the oligonucleotide from Z.
